(19) United States
(12) Patent Application Publication
(10) Pub. No.: US 2019/0345440 A1
(43) Pub. Date: Nov. 14, 2019

(54) Device for Preparing Stem Cell Spheroids, Method for Preparing Stem Cell spheroids and Method for Preserving Stem Cells

(71) Applicant: UNIVERSITY OF MACAU, Macau (CN)

(72) Inventors: Ren-He XU, Macau (CN); Bin JIANG, Macau (CN); Enqin LI, Macau (CN)

(73) Assignee: UNIVERSITY OF MACAU, Macau (CN)

(21) Appl. No.: 16/221,659

(22) Filed: Dec. 17, 2018

(30) Foreign Application Priority Data
May 10, 2018 (CN) 201810441644.2

(51) Int. Cl. C12N 5/00; C12N 5/0775; C12N 5/0735; C12N 5/074; C12M 1/32

(52) U.S. Cl. CPC C12N 5/0075; C12N 5/0663; C12N 5/0668; C12N 2513/00; C12N 5/0696; C12M 23/12; C12N 5/0606

(57) ABSTRACT

Disclosed are a device for preparing stem cell spheroids, a method for preparing stem cell spheroids and a method for preserving stem cells which relate to the technical field of preservation and transportation of stem cells. The device for preparing stem cell spheroids disclosed herein includes a substrate. The substrate is provided thereon with culture chambers, each of which has side walls and a bottom wall made of a material incompatible with stem cells. By culturing stem cells with this device, the stem cells may be made into spheroids and thus form stem cell spheroids. The device may be used as a means of preserving or transporting stem cells which makes it possible for stem cells to still have a higher viability and pluripotency even after a long time of preservation, storage and transportation under ambient temperature condition in the form of spheroids.

### CROSS-REFERENCE TO RELATED APPLICATIONS

The present application claims the priority of the Chinese patent application No. 201810441644.2, filed with the Chinese Patent Office on May 10, 2018, and entitled “Device for Preparing Stem Cell Spheroids, Method for Preparing Stem Cell Spheroids and Method for Preserving Stem Cells”, which is incorporated herein by reference in its entirety.

### BACKGROUND OF THE DISCLOSURE

### 1. Field of the Disclosure

The present disclosure relates to the technical field of stem cell preservation and transportation, and specifically to a device for preparing stem cell spheroids, a method for preparing stem cell spheroids and a method for preserving stem cells.

### 2. Field of the Related Art

Mammalian cells generally are cultured in a humidified incubator at 37° C. with 5% CO₂, 20% O₂ and an appropriate medium. Deviation from such standard condition might alter the cell functions or even lead to cell abnormality or death. Human embryonic stem cells (hESCs), induced pluripotent stem cells (iPSCs) and their progenies are more likely to be affected by inappropriate culturing conditions. When changes happen to any standard parameters, spontaneous differentiation, karyotype change, cell exfoliation or death might occur in these cells.

Many kinds of cells can be stored under refrigerated or ambient temperature condition for only a short time (1-2 days). The term “ambient temperature condition (AC)” as used herein refers to a condition with no standard levels of CO₂ and O₂ at ambient temperature in a sealed container, where the medium is not replaced. After a short time of storage under AC, the cell viability decreases significantly. Therefore, a long-term storage and a long-distance transportation of cells require cryopreservation which restricts the study and treatment application of stem cells. Despite its inconvenience and high cost, cryopreservation has always been an indispensable method for storing and transporting cells, and people rarely try to modify or simplify it.

Recently emerging 3D cell culturing and printing also relate to cell treatment at ambient temperature. This process usually turns isolated cells and biocompatible supporting materials into tissue blocks which are used in regenerative medicine. But decreased cell viability is the main challenge for this process and subsequent transplant.

### SUMMARY

A purpose of the present disclosure is to provide a device for preparing stem cell spheroids. By culturing stem cells with such device, stem cells may be made into spheroids and form stem cell spheroids. This device may be used as a means of preserving or transporting stem cells, which makes it possible for stem cells to have a higher viability and pluripotency even after a long time of preservation, storage and transportation under ambient temperature condition in the form of spheroids.

Another purpose of the present disclosure is to provide a method for preparing stem cell spheroids. Stem cell spheroids may be prepared by such method, and the prepared stem cell spheroids may have a higher viability and pluripotency even after a long time of preservation, storage and transportation under ambient temperature condition.

A further purpose of the present disclosure is to provide a method for preserving stem cells. Stem cells may be made into spheroids by such method, and so they exist in the form of spheroids so that they may have a higher viability and pluripotency even after a long time of preservation and transportation under ambient temperature condition.

### BRIEF DESCRIPTION OF THE DRAWINGS

In order to more clearly illustrate the technical solutions provided in the embodiments of the present disclosure, drawings necessary for the embodiments will be briefly described below. It will be appreciated that the following drawings merely show some embodiments of the disclosure and thus should not be construed as limiting the scope. Other related drawings can be obtained by those ordinarily skilled in the art according to these drawings without paying any creative effort.

FIG. 1 shows the result of cell viability assay of MSCs after standing as spheroids under ambient temperature (AC) condition for 7 days in Example 1 of the present disclosure.

FIG. 2 shows the result of morphology assay of MSCs after standing as spheroids under ambient temperature (AC) condition for 7 days in Example 1 of the present disclosure.

FIG. 3 shows the viabilities of MSCs after standing as spheroids under ambient temperature (AC) condition for 7 and 9 days in Example 1 of the present disclosure.

FIG. 4 shows the results of flow cytometry assay of MSCs for AnnexinV and dead cell marker (PI) after standing as spheroids under ambient temperature (AC) condition for 7 days in Example 1 of the present disclosure.

FIG. 5 shows the results of assay for morphological and biological characteristics of MSCs after standing as spheroids under ambient temperature (AC) condition for 7 days in Example 2 of the present disclosure.

FIG. 6 shows the results of assay for immune response and regulation of MSCs after standing as spheroids under ambient temperature (AC) condition for 7 days in Example 2 of the present disclosure.

FIG. 7 is a structural schematic perspective diagram of a device for preparing stem cell spheroids in Embodiment 1 of the present disclosure.

FIG. 8 is a structural schematic diagram of a culture chamber of the device for preparing stem cell spheroids in Embodiment 1 of the present disclosure.

FIG. 9 is a structural schematic diagram of the bottom wall of the culture chamber of the device for preparing stem cell spheroids in Embodiment 1 of the present disclosure.

FIG. 10 is a sectional structural schematic diagram of the device for preparing stem cell spheroids in Embodiment 1 of the present disclosure.

FIG. 11 is a structural schematic diagram of the culture chamber of the device for preparing stem cell spheroids in Embodiment 1 of the present disclosure when filled with single-cell suspension.

FIG. 12 is a structural schematic diagram of the device for preparing stem cell spheroids in Embodiment 1 of the present disclosure when stem cells are forming spheroids.

FIG. 13 is a structural schematic diagram of the device for preparing stem cell spheroids in Embodiment 1 of the present disclosure when its surface is plastic-packaged with a sealing membrane.

FIG. 14 is a structural schematic diagram of a culture flask for preparing stem cell spheroids in Embodiment 2 of the present disclosure when horizontally placed.

FIG. 15 is a structural schematic diagram of the culture flask for preparing stem cell spheroids in Embodiment 2 of the present disclosure when vertically placed.

FIG. 16 is a reference flow diagram of using the device for preparing stem cell spheroids provided in Embodiment 1 and using the culture flask for preparing stem cell spheroids provided in Embodiment 2.

Reference signs: 10-device for preparing stem cells; 11-substrate; 12-culture chamber; 13-side wall; 14-bottom wall; 15-recess; 16-sealing membrane; 20-single-cell suspension; 21-stem cell spheroid; 22-culture flask; 23-flask body; 24-flask mouth.

### DETAILED DESCRIPTION

To make the purposes, technical solutions and advantages of the present disclosure more clear, the technical solutions in the embodiments of the present disclosure will be clearly and completely described below. Embodiments for which no specific condition is indicated should be done under conventional conditions or conditions recommended by the manufacturer. All those agents or instruments for which no manufacturer is indicated are all conventional products which are commercially available.

The present disclosure is implemented in the following way:

In one aspect, some embodiments of the present disclosure provide a device for preparing stem cell spheroids, which includes a substrate. The substrate is provided with culture chambers thereon. Each of the culture chambers has side walls and a bottom wall made of a material incompatible with stem cells.

During stem cell culture, the surrounding microenvironment has a significant influence on spheroidal formation of stem cells. Especially, adherent culture of stem cells is not beneficial to spheroidal formation. The device for preparing stem cell spheroids provided by the embodiments of the present disclosure has culture chambers with side walls and bottom walls made of a material incompatible with stem cells. As the side walls and the bottom walls are made of a material incompatible with stem cells, the side walls and the bottom walls are not compatible with stem cells and thus form a microenvironment incompatible with stem cells which prevents adherent culture of stem cells, and facilitate stem cells to aggregate into masses and form stem cell spheroids during proliferation.

And this device may be used as a means of preserving or transporting stem cells which makes it possible for stem cells to have a higher viability and pluripotency even after a long time of preservation, storage and transportation under ambient temperature condition in the form of spheroids.

Further, in some embodiments of the present disclosure, the material includes but is not limited to materials of celluloid, polyvinyl chloride (PVC), natural resin, etc.

Celluloid, polyvinyl chloride and natural resin are all not compatible with stem cells. All the side walls and the bottom walls of the culture chambers made of such materials can form a microenvironment which is not compatible with stem cells and facilitates stem cells to form spheroids and thus form stem cell spheroids during proliferation.

Preferably, in some embodiments of the present disclosure, polyvinyl chloride is used to make the side walls and the bottom walls of the culture chambers, so as to improve the ability of stem cells to form spheroids by further facilitating them to form spheroids and thus form stem cell spheroids during proliferation.

Sure, in some embodiments of the present disclosure, the entire device may be also made of materials incompatible with stem cells (e.g. materials of celluloid, polyvinyl chloride, natural resin, etc.). Accordingly, the side walls and the bottom walls of the culture chambers are also made of materials incompatible with stem cells, and thus likewise can form a microenvironment which facilitates spheroidal formation of stem cells and which is incompatible with stem cell spheroids.

In this case, the natural resin includes but is not limited to materials of rosin, amber or lac which are all not compatible with stem cells.

In some embodiments of the present disclosure, it may also be the case where the side walls and the bottom walls of the culture chambers are coated with a film made of a material incompatible with stem cells. Such structural design can also form a microenvironment which is incompatible with stem cell spheroids, prevent adherent growth of cells, and facilitates spheroidal formation during reproduction of cells. Such structure design also falls within the scope of protection of the present disclosure.

In a word, all containers, e.g. culture flasks, cavities or chambers, made of any material not compatible with stem cells or even a low-absorption specific material e.g. agarose which forms a microenvironment incompatible with stem cell spheroids fall within the scope of protection of the present disclosure.

Further, in some embodiments of the present disclosure, the bottom walls of the culture chambers are provided with recesses.

During progressive aggregation of stem cells into spheroids, they tend to settle and come into contact with the bottom wall as a result of gravity, etc. Provision of a plurality of recesses on the bottom wall makes it possible for the stem cells to be uniformly distributed and form similarly sized spheroids. In this way, it is possible to control the size of stem cell spheroids by adjusting the cell density at the time of filling stem cell.

Without recess, formation of stem cell spheroids is also possible, but a recess design makes it possible to obtain similarly sized stem cell spheroids.

The recess may be shaped as required. Preferably, the recess is U-shaped like a semicircle, V-shaped like a circular cone or is a four-side inverted pyramid.

Further, in some embodiments of the present disclosure, the recess has a depth of 0.8-1.2 mm, preferably 1 mm.

Further, in some embodiments of the present disclosure, there are a plurality of culture chambers.

Preferably, there are 9 culture chambers.

Preferably, the 9 culture chambers are arranged on the substrate at a uniform interval in a 3×3 array.

Preferably, the spacing between any adjacent two of the culture chambers is 0.25-0.35 cm.

Preferably, each of the culture chambers has a cubic structure with a length of 1.4-1.6 cm, a width of 1.4-1.6 cm and a depth of 0.4-0.6 cm.

In another aspect, the embodiments of the present disclosure provide a method for preparing stem cell spheroids which includes placing stem cell suspension in the culture chambers in the device as described above for suspension culture.

Placing stem cells and culture solution in the culture chambers in the device as described above for suspension culture prevents adherent growth and facilitates spheroidal formation by utilizing the property of the culture chambers that they are incompatible with stem cells on one hand, and causes stem cells to aggregate into stem cell masses spontaneously and thus form stem cell spheroids by utilizing the high expression of cell adhesion factor N/E-Cadherin on surfaces of stem cells, on the other hand.

The stem cell spheroids prepared by such method may have a higher viability and pluripotency even after a long time of preservation, storage and transportation under ambient temperature condition.

Further, in some embodiments of the present disclosure, the above stem cells are single-cell suspension.

Further, in some embodiments of the present disclosure, the stem cells are cultured in the culture chambers at a density of 0.5-2×10⁶/ml for 24-48 hours.

Further, in some embodiments of the present disclosure, the culture solution contains DMEM low glucose medium, 18-22% of fetal calf serum or DMEM serum replacement, 0.8-1.2% of non-essential amino acids and 4.8-5.2% of L-glutamine.

DMEM low glucose medium refers to DMEM medium containing about 1.5 g/L of glucose.

Further, in some embodiments of the present disclosure, the stem cells are mesenchymal stem cell strains formed by differentiation of human pluripotent stem cell strains or mesenchymal stem cells separated from an adult issue.

Preferably, in some embodiments of the present embodiment, the adult tissue includes but is not limited to bone marrow, fat and umbilical cord blood.

In a further aspect, the embodiments of the present disclosure provide a method for preserving stem cells, which includes placing stem cell suspension in the culture chambers in the device as described above for suspension culture so that the stem cells will form spheroids.

The culture chambers in the device are plastic-packaged with aluminum foils.

Such method for preserving stem cells enables storage and transportation with such device at ambient temperature directly. The stem cell spheroids maintain a viability of not less than 90% and excellent biological functions within 10 days.

It should be noted that in the case of culture flask, one may simply tighten the flask cap. Then, they may be stored and transported at ambient temperature.

Further, in some embodiments of the present disclosure, stem cell medium is added to the culture chambers until it reaches 90% of the volume of the culture chambers, and then the culture chambers in the device are plastic-packaged with aluminum foils under aseptic condition.

This preservation method is used to make stem cells form spheroids so that stem cells are present in the form of spheroids and thus they may have a higher viability and pluripotency even after a long time of preservation and transportation under ambient temperature condition.

The characteristics and effects of the present disclosure will be further described in details below in combination with the embodiments.

### EMBODIMENT 1

As shown in FIGS. 7-10, the device 10 for preparing stem cell spheroids provided in the present embodiment includes a substrate 11 made of polyvinyl chloride which is a material incompatible with stem cells.

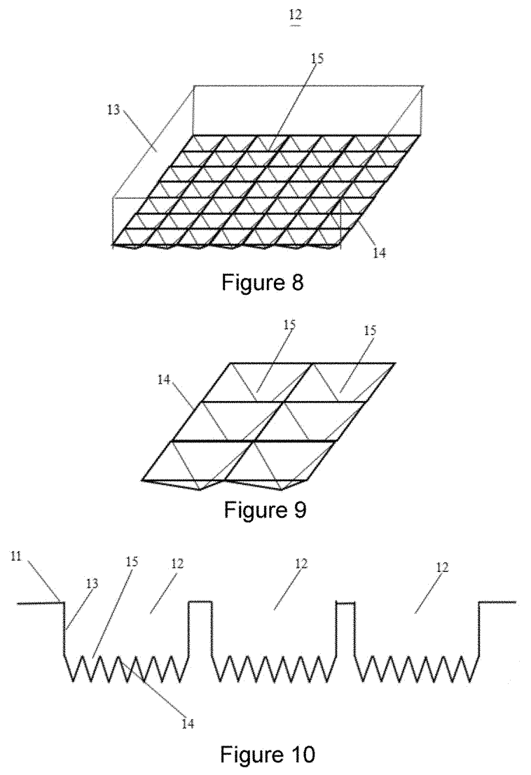

The substrate 11 is provided thereon with culture chambers 12. Each of the culture chambers 12 has side walls 13 and a bottom wall 14 (see FIG. 7 and FIG. 8).

The bottom 14 has a plurality of recesses 15. In the present embodiment, the recesses 15 are shaped as inverted pyramids (see FIGS. 7, 8 and 9).

In the present embodiment, the number and size of the culture chambers 12 and the size of the recesses are designed with reference to the following parameters.

There are 9 culture chambers which are arranged on the substrate at a uniform interval in a 3×3 array. The side walls of the culture chambers have a height (i.e. depth, excluding the depth of the recess) of about 0.5 cm. The spacing between the culture chambers is about 0.3 cm. The culture chambers have a length and width of both about 1.5 cm.

The recesses 15 have a depth of about 1 mm. The recesses 15 shaped as inverted pyramids have a length and width of both about 15 mm.

It should be noted that in other embodiments, the number and size of the culture chambers and the size of the recesses may be designed as practical requirement.

In addition, it should also be noted that in other embodiments, the recesses may be U-shaped like a semicircle or V-shaped like a circular cone or flat-bottomed (i.e. having a section of inverted trapezoid).

Specifically, the method for preparing stem cell spheroids using the device provided in the present embodiment includes:

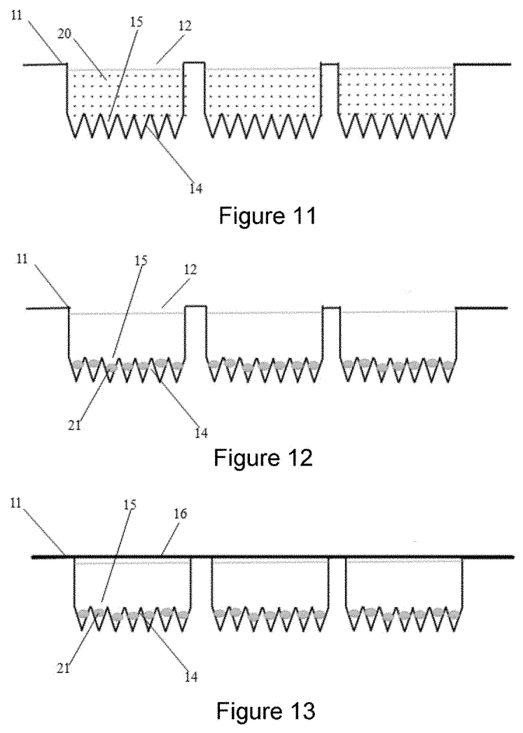

mixing human mesenchymal stem cells (MSCs) with culture solution to obtain single-cell suspension 20, filling the culture chambers 12 in the device with the single-cell suspension for suspension culture (see FIG. 11), keeping the stem cells in the culture chambers at a density of 1×10⁶/ml and culturing for 36 hours. In this way, a 3D suspension culture condition is achieved utilizing the property of polyvinyl chloride that it is incompatible with cells. And stem cells spontaneously settle in the recesses with the help of the high expression of cell adhesion factor N/E-Cadherin, on the surface of the stem cells, and thus they aggregate to form stem cell spheroids 21 (see FIG. 12).

The stem cell spheroids may be used directly after collected, or used after they are digested into single cells by trypsin.

The culture solution contains DMEM low-glucose medium, 20% of fetal calf serum, 1% of non-essential amino acids and 5% of L-glutamine.

It should be noted that in other embodiments, the stem cells may be mesenchymal stem cell strains formed by differentiation of human pluripotent stem cell strains or mesenchymal stem cells separated from an adult issue. The adult tissue includes but is not limited to bone marrow, fat and umbilical cord blood.

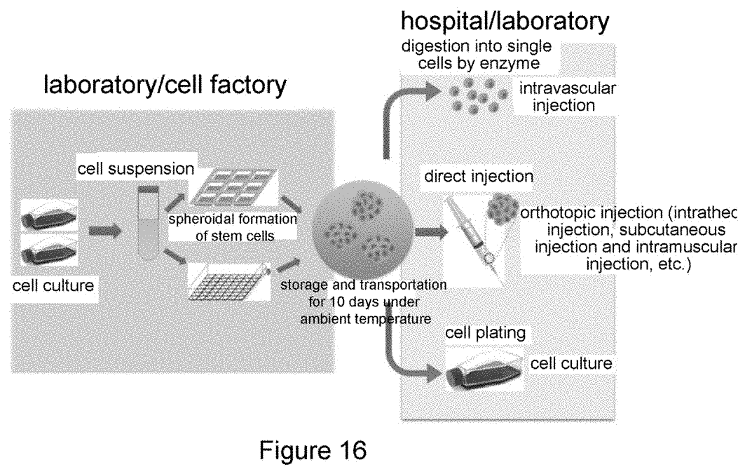

Besides, the device may be used as a means of preserving or transporting stem cells. Its use procedure is shown in FIG. 16. For example, the surface of the substrate 11 is plastic-packaged with a sealing membrane 16, e.g. aluminum foil or plastic membrane, after filled with stem cell suspension (see FIG. 13), so that the stem cells have a higher viability and pluripotency even after being preserved, stored and transported under ambient temperature condition for a long time (e.g. 7 to 10 days) in the form of spheroids. In use, they are digested into single cells by enzyme and then used for intravascular injection, direct injection or cell culture again.

### EMBODIMENT 2

The culture bottom 22 for preparing stem cell spheroids provided in the present embodiment is made by encapsulating the substrate 11 provided in Embodiment 1 within the flask body 23. The culture flask 22 includes a flask body 23 and a flask mouth 24. The flask mouth 24 is communicated with the cavity within the flask body 23. Overall, the flask body 23 has a square structure and the flask body 23 is made of polyvinyl chloride which is a material incompatible with stem cells. One of the six side walls of the flask body 23 is made by the substrate 11 in Embodiment 1. The flask mouth 24 is provided on any one of the other five side walls. Unlike Embodiment 1, there is one culture chamber on the substrate 11 used in the present embodiment and the cavity within the flask body 23 is the culture chamber.

Sure, in other embodiments, a plurality of substrates may be stacked in the flask body 23 and the number of stem cell spheroids may be increased within preset space. Sure, the number of substrates may be arranged as practical requirement.

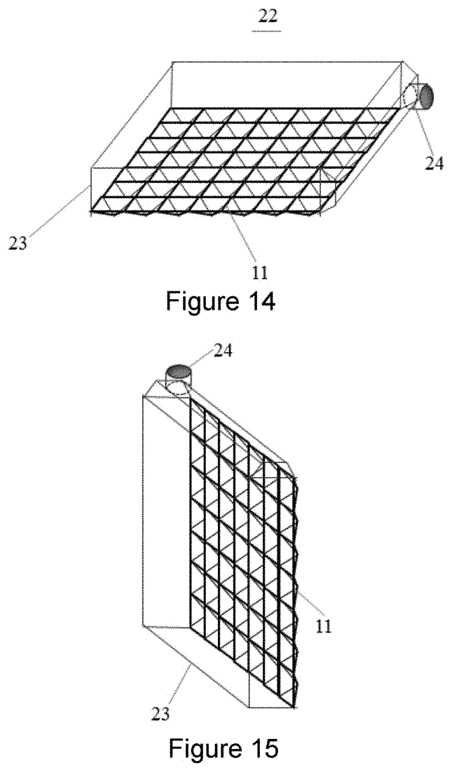

The culture flask for preparing stem cell spheroids provided in the present embodiment does not only has the effects provided in Embodiment 1, but also is more convenient and safer when used to preserve or transport stem cells. Its use method is shown in FIG. 16. The flask mouth is sealed after stem cell suspension is filled. Then the flask is laid horizontally (see FIG. 14) in a way that the plane where the substrate is located is substantially parallel with the horizontal plane, so that the stem cells fully settle in the recesses on the substrate and form cell spheroids. And thereby the stem cells may be preserved, stored and transported under ambient temperature condition for a long time (e.g. 7 to 10 days) in the form of spheroids. In use, the culture flask may be vertically placed (see FIG. 15). In this way, the cell spheroids on the substrate may aggregate at the bottom of the culture flask, making it easy to collect them. Then the stem cell spheroids are taken out via the flask mouth by a suitable tool e.g. an syringe, for use in subsequent steps.

### EXAMPLE 1

1. An experiment method by using AO/PI staining method to detect the cell viability of MSCs after standing under ambient temperature (AC) condition as spheroids, includes:
taking MSCs, forming spheroids (Sp) by the method of Embodiment 1, transferring into a centrifuge tube, standing for 7 days under ambient temperature condition, with the corresponding spherical cells being named as EMSC_{Sp-AC/D7}, then digesting EMSC_{Sp-AC/D7} into single cells and detecting for live/dead cells by AO/PI staining;
digesting EMSC_{Sp-AC/D7} into single cells and re-plating them in a culture dish to form monolayer cells again, then standing for 7 days under normal condition, with the corresponding cells being named as EMSC_{Sp-AC/D7-ML}, then detecting for live/dead cells directly by AP/PI staining; and
using EMSCs cultured in monolayers (MLs) in a 6-well plate as control, standing under ambient temperature condition for 7 days, with the obtained cells being named as EMSC_{ML-AC/D7}, and then detecting for live/dead cells directly by AI/PO staining.

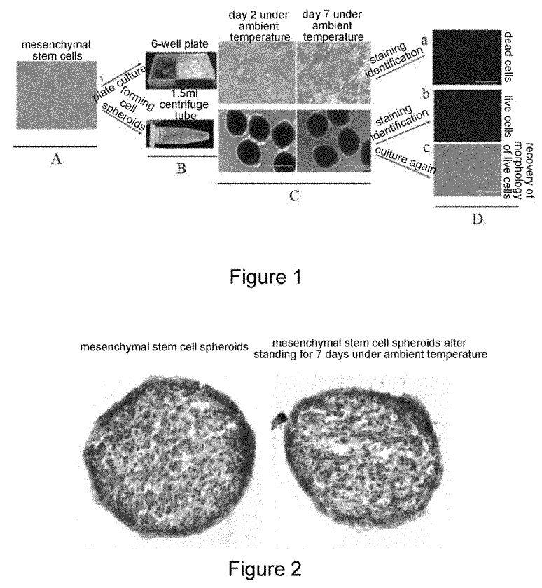

The result is shown in FIG. 1.

FIG. 1 shows the viability of MSCs after standing as spheroids under ambient temperature (AC) condition for 7 days.

In FIG. 1:
A is the morphology of mesenchymal stem cells under normal culture condition, with a scale bar of 400 μm;
B is a treatment way of standing under ambient temperature;
C shows the morphologies at different time when standing under ambient temperature, with a scale bar of 400 μm; and
D shows the cell viability and morphology after being treated under ambient temperature, with a scale bar of 400 μm, wherein a is the result of staining of EMSC_{ML-AC/D7}; b is the result of staining of EMSC_{Sp-AC/D7}; and c is EMSC_{Sp-AC/D7-ML}.

As can be seen from the result in FIG. 1, there are obviously more green fluorescent spots in EMSC_{Sp-AC/D7} from MSCs after standing as spheroids under ambient temperature (AC) condition for 7 days. This indicates more live cells (FIG. 1-D) and demonstrates that stem cells have a longer viable period if they are present or preserved as spheroids.

2. Detection of morphology of MSC spheroids, is conducted by:
taking MSCs, forming MSC spheroids by the method of Embodiment 1, standing under ambient temperature (AC) condition for 7 days, and detecting the morphologies of the spheroids before and after culture by H&E staining, wherein the result is shown in FIG. 2.

As can be seen from the result in FIG. 2, the morphology of the MSC spheroids is intact after standing under ambient temperature (AC) condition for 7 days and shows no obvious difference from the morphology of the cultured MSC spheroids.

3. Detection of the viability of MSC spheroids after standing under ambient temperature (AC) condition for 7 and 9 days, is conducted by:
taking MSCs, forming MSC spheroids by the method of Embodiment 1, culturing under ambient temperature (AC) condition for 9 days, taking samples at day 7 and day 9, respectively, determining the proportion of live cells (AO+/PI−) by flow cytometry; setting a spheroid group, a control group, a dissociation group and a plate group, respectively;
for the spheroid group, taking MSCs, forming MSC spheroids by the method of Embodiment 1, culturing under ambient temperature (AC) condition for 9 days, taking samples at day 7 and day 9, respectively, and determining the proportion of live cells (AO+/PI−) by flow cytometry;
for the control group, determining the viability of mesenchymal stem cells cultured under normal condition;
for the disassociation group, digesting the mesenchymal stem cells cultured under normal condition into single cells, standing under ambient temperature and then determining the viability; and
for the plate group: allowing the mesenchymal stem cells cultured under normal condition to directly stand under ambient temperature by the way as shown in FIG. 1B (6-well plate) and then determining the viability.

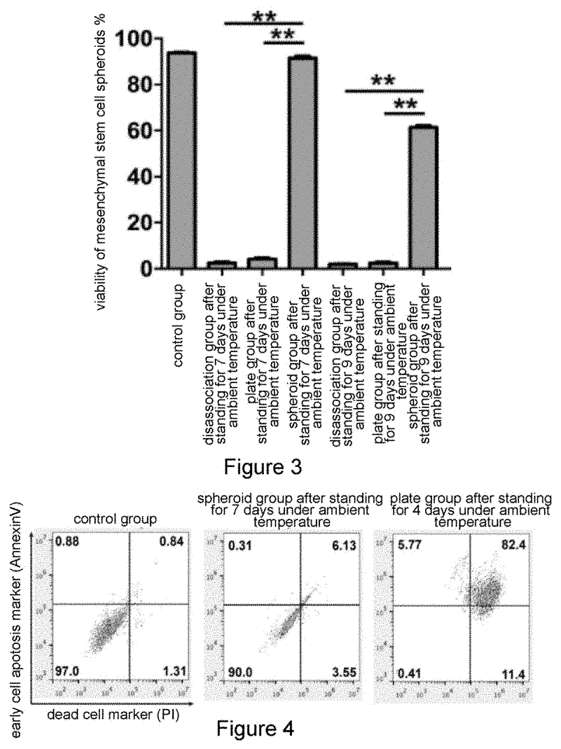

The result is shown in FIG. 3, in which the data is expressed as mean value±standard deviation (n=3), with **: t test P<0.01.

As can be seen from FIG. 3, either in the case of standing under ambient temperature for 7 days or in the case of standing under ambient temperature for 9 days, the mesenchymal stem cells in the spheroid group have an obviously higher cell viability than the other groups.

4. Detection of MSCs for early cell apotosis marker i.e. AnnexinV and dead cell marker (PI), is conducted by:
setting a spheroid group, a control group and a plate group, for the spheroid group, taking MSCs, forming MSC spheroids by the method of Embodiment 1, standing under ambient temperature (AC) condition for 7 days, and detecting for AnnexinV and dead cell marker (PI) on their EMSCs;
for the control group, determining the viability of the mesenchymal stem cells cultured under normal condition; and
for the plate group, allowing the mesenchymal stem cells cultured under normal condition to directly stand under ambient temperature by the way as shown in FIG. 1B (6-well plate) and then determining the viability.

The detection results are shown in FIG. 4. As can be seen, in the spheroid group, the contents of AnnexinV and dead cell marker (PI) are lower, which indicates that the cells in the spheroid group have a lower cell death rate than the plate group when standing under ambient temperature.

### EXAMPLE 2

MSC_{Sp-AC-ML} cells kept the morphological and biological characteristics of MSCs.

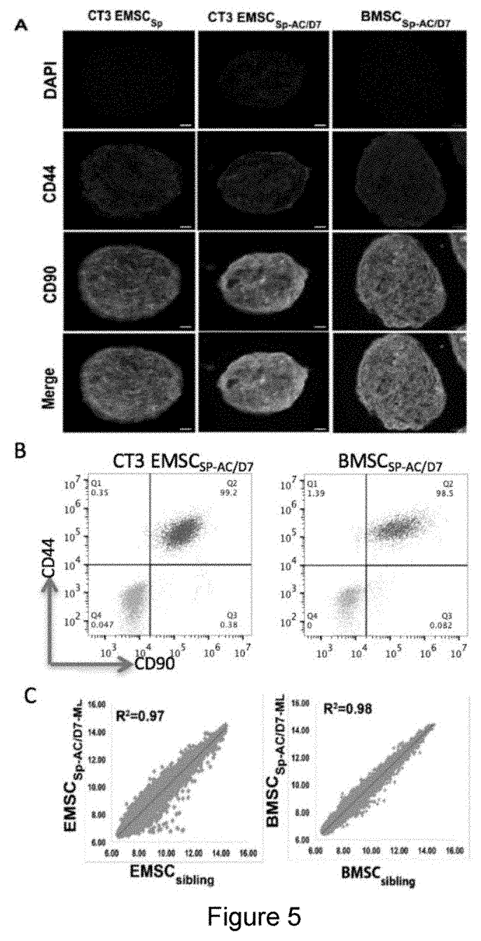

FIG. 5 shows that MSC_{Sp-AC-ML} cells keep the morphological and biological characteristics of MSCs. (A) Immunostaining for CD902 and CD44 (red) in frozen sections of MSC spheroids with or without spheroidal formation and exposed under AC is conducted. The cell nucleuses were counterstained with DAPI (blue). The scale bar is 100 μm. Cells positive for both CD90 and CD44 appear as red spots with blue isotype control in flow cytometry diagram. (B) Comparison of microarray-based gene expression profiles of MSC samples (EMSC_{Sp-AC/D7-ML} and BMSC_{Sp-AC/D7-ML}) recovered under AC with their corresponding sibling controls (maintained in normal monolayer cultures). R2 represents correlation coefficient.

### EXAMPLE 3

The EMSC spheroids prepared by the method of Embodiment 1 were recovered after they had stood for 7 days. They were detected for immune response and regulation and influence on proliferation of mouse lymphocytes.

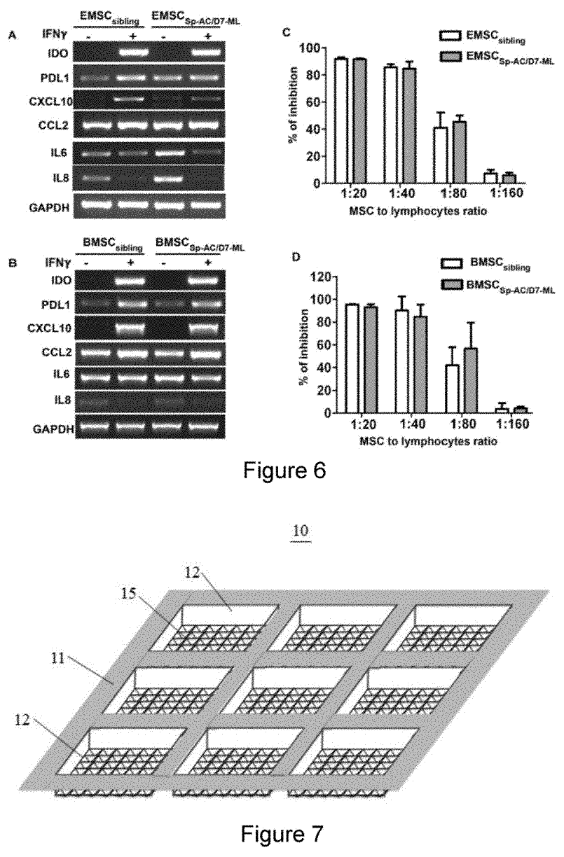

1. RT-PCR was used to analyze the expressions of representative inflammatory genes (IDO, PDL1, CXCL10, CCL2, IL6 and IL8) in EMSC_{sibling}, EMSC_{sp-AC/D7-ML}, BMSC_{sibling} and BMSC_{sp-AC/D7-ML} after being treated by 20 ng/ml IFNγ for 24 hours or not treated. The results are shown in A and B in FIG. 6.

As can be seen from A and B in FIG. 6, both EMSC_{sp-AC/D7} and BMSC_{sp-AC/D7} show a good immune response.

2. Detection of influence of EMSC_{sp-AC/D7} on proliferation of mouse lymphocytes.

CSFE dilution method was used to detect the influence of EMSC_{sp-AC/D7-ML}, EMSC_{sibling}, BMSC_{sibling}, BMSC_{sp-AC/D7 ML} on the proliferation of mouse lymphocytes after they were mixed with mouse lymphocytes in different ratios. The results are shown in C and D in FIG. 6 (in FIG. C, 1:20 is the ratio of EMSC_{sp-AC/D7} or EMSC_{sibling} to the number of mouse lymphocytes in the culture system, and the data is expressed as mean value±SD (n=3)).

As can be seen from C and D in FIG. 6, EMSC_{sp-AC/D7} and BMSC_{sp-AC/D7} have a higher inhibition ratio under a condition of 1:80.

In conclusion, the study of the present disclosure shows that stem cells after forming spheroids are also tolerant to low temperature (e.g. room temperature) and are more viable than cells cultured in monolayers. Most monolayer MSCs are dead after 7-9 days of storage under ambient temperature, whereas MSC spheroids still remain highly viable. Monolayer ESCs are dead already after 4 days of storage under ambient temperature condition, whereas ESC spheroids still remain highly viable.

MSCs have been proven in animal models and clinical trials to be effective in treating many autoimmune diseases, inflammations and degenerative diseases. Single cells separated from living MSCs which were preserved under ambient temperature as cell spheroids, are still effective in treating mouse model of colitis by injection. Therefore, the study result of the present disclosure is of an important significance in preserving stem cells under ambient temperature and it contributes to its fundamental research, mass production and long-distance transportation for clinic treatment.

The above description only shows the preferable embodiments of the present disclosure and is not intended to limit the present disclosure. Various modifications and variations of the present disclosure will occur to those skilled in the art. Any modifications, equivalent replacements and improvements made within the spirit and principle of the present disclosure shall be encompassed by the scope of protection of the present disclosure.

What is claimed is:

1. A device for preparing stem cell spheroids, the device comprising:
a substrate, wherein the substrate is provided thereon with at least one culture chamber that has side walls and a bottom wall made of a material incompatible with stem cells.

2. The device according to claim 1, wherein the material is selected from the group consisting of: celluloid, polyvinyl chloride and natural resin.

3. The device according to claim 1, wherein the bottom wall of the at least one culture chamber has a plurality of recesses.

4. The device according to claim 2, wherein the bottom wall of the at least one culture chamber has a plurality of recesses.

5. The device according to claim 3, wherein each of the recesses has a depth of 0.8 to 1.2 mm.

6. The device according to claim 4, wherein each of the recesses has a depth of 0.8 to 1.2 mm.

7. The device according to claim 1, wherein the at least one culture chamber are more than one in number;
spacing between any adjacent two of the at least one culture chamber is 0.25-0.35 cm; and
each of the at least one culture chamber has a cubic structure with a length of 1.4-1.6 cm, a width of 1.4-1.6 cm and a depth of 0.4-0.6 cm.

8. The device according to claim 2, wherein the at least one culture chamber are more than one in number;
spacing between any adjacent two of the at least one culture chamber is 0.25-0.35 cm; and
each of the at least one culture chamber has a cubic structure with a length of 1.4-1.6 cm, a width of 1.4-1.6 cm and a depth of 0.4-0.6 cm.

9. A method for preparing stem cell spheroids, the method comprising:
placing stem cells and culture solution in the at least one culture chamber of the device according to claim 1 for suspension culture.

10. The method according to claim 9, wherein the stem cells are cultured in the at least one culture chamber at a density of 0.5 to 2×10⁶/ml for 24 to 48 hours.

11. The method according to claim 10, wherein the culture solution contains DMEM low-glucose medium, 18 to 22% of fetal calf serum or serum replacement, 0.8 to 1.2% of non-essential amino acids and 4.8 to 5.2% of L-glutamine.

12. The method according to claim 10, wherein the stem cells are mesenchymal stem cell strains formed by differentiation of human pluripotent stem cell strains or mesenchymal stem cells separated from an adult issue.

13. The method according to claim 11, wherein the stem cells are mesenchymal stem cell strains formed by differentiation of human pluripotent stem cell strains or mesenchymal stem cells separated from an adult issue selected from the group consisting of: bone marrow, fat, and umbilical cord blood.

14. A method for preserving stem cell spheroids, the method comprising:
placing a stem cell suspension in the at least one culture chamber of the device according to claim 1 for suspension culture so that the stem cells form spheroids,
wherein the at least one culture chamber is plastic-packaged with aluminum foils.

15. The method according to claim 14, wherein the material is at least one material selected from the group consisting of: celluloid, polyvinyl chloride, and natural resin.

16. The method according to claim 14, wherein the bottom wall of each of the at least one culture chamber has a plurality of recesses.

17. The method according to claim 14,
wherein the material is at least one material selected from the group consisting of: celluloid, polyvinyl chloride, and natural resin; and
the bottom wall of each of the at least one culture chamber has a plurality of recesses.

18. The method according to claim 14,
wherein the bottom wall of the at least one culture chamber has a plurality of recesses; and
each of the recesses has a depth of 0.8-1.2 mm.

19. The method according to claim 14, wherein the at least one culture chamber are more than one in number;
spacing between any adjacent two of the at least one culture chamber is 0.25-0.35 cm; and
each of the at least one culture chamber has a cubic structure with a length of 1.4-1.6 cm, a width of 1.4-1.6 cm and a depth of 0.4-0.6 cm.

20. The method according to claim 14, wherein
the material is at least one material selected from the group consisting of celluloid, polyvinyl chloride and natural resin;
the at least one culture chamber are more than one in number;
spacing between any adjacent two of the at least one culture chamber is 0.25-0.35 cm; and
each of the at least one culture chamber has a cubic structure with a length of 1.4 to 1.6 cm, a width of 1.4 to 1.6 cm and a depth of 0.4 to 0.6 cm.
